(19) United States
(12) Patent Application Publication
(10) Pub. No.: US 2005/0053957 A1
(43) Pub. Date: Mar. 10, 2005

(54) Polynucleotide sequence detection assays

(75) Inventor: Barnett B. Rosenblum, San Jose, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 10/718,016

(22) Filed: Nov. 19, 2003

Related U.S. Application Data
(60) Provisional application No. 60/427,818, filed on Nov. 19, 2002.
(60) Provisional application No. 60/445,636, filed on Feb. 7, 2003.

(51) Int. Cl. C12Q 1/68; C07H 21/04

(52) U.S. Cl. 435/6; 536/24.3

(57) ABSTRACT

The present teachings relate to methods, compositions, and kits for detecting one or more target polynucleotide sequences in a sample. In some embodiments of the present teachings, oligonucleotides are hybridized to complementary target polynucleotides and are ligated together to form a ligation product. In some embodiments of the present teachings, the ligation product can be amplified, and the identity and quantity of the target polynucleotides determined based on sequence introduced in the ligation reaction. Some embodiments of the present teachings allow for the detection of target polynucleotide sequences by hybridization of a mobility probe to sequence information introduced in the ligation reaction. Some embodiments of the present teachings provide for highly multiplexed detection, identification, and quantification of a plurality of target polynucleotides using a variety of analytical procedures.

### CROSS-REFERENCE TO RELATED APPLICATIONS

This application claims the benefit under 35 USC § 119(e) of U.S. Provisional Application No. 60/427,818, filed Nov. 19, 2002 and U.S. Provisional Application No. 60/445,636, filed Feb. 7, 2003, each of which are assigned to the assignee hereof, and each of which are expressly incorporated herein by reference in their entireties.

The present invention relates to methods for detecting one or more polynucleotide sequences in one or more samples, and to reagents and kits for use therein.

Methods for detection and analysis of target nucleic acids have found wide utility in basic research, clinical diagnostics, forensics, and other areas. One important use is in the area of genetic polymorphism. Genetic polymorphisms generally concern the genetic sequence variations that exist among homologous loci from different members of a species. Genetic polymorphisms can arise through the mutation of genetic loci by a variety of processes, such as errors in DNA replication or repair, genetic recombination, spontaneous mutations, transpositions, etc. Such mutations can result in single or multiple base substitutions, deletions, or insertions, as well as transpositions, duplications, etc.

Single base substitutions (transitions and transversions) within gene sequences can cause missense mutations and nonsense mutations. In missense mutations, an amino acid residue is replaced by a different amino acid residue, whereas in nonsense mutations, stop codons are created that lead to truncated polypeptide products. Mutations that occur within signal sequences, e.g., for directing exon/intron splicing of mRNAs, can produce defective splice variants with dramatically altered protein sequences. Deletions, insertions, and other mutations can also cause frameshifts in which contiguous residues encoded downstream of the mutation are replaced with entirely different amino acid residues. Mutations outside of exons can interfere with gene expression and other processes.

Genetic mutations underlie many disease states and disorders. Some diseases have been traced directly to single point mutations in genomic sequences (e.g., the A to T mutation associated with sickle cell anemia), while others have been correlated with large numbers of different possible polymorphisms located in the same or different genetic loci (e.g., cystic fibrosis). Mutations within the same genetic locus can produce different diseases (e.g., hemoglobinopathies). In other cases, the presence of a mutation may indicate susceptibility to particular condition for a disease but is insufficient to reliably predict the occurrence of the disease with certainty. Most known mutations have been localized to gene-coding sequences, splice signals, and regulatory sequences. However, it is expected that mutations in other types of sequences can also lead to deleterious, or sometimes beneficial, effects.

The large number of potential genetic polymorphisms poses a significant challenge to the development of methods for identifying and characterizing nucleic acid samples and for diagnosing and predicting disease. In other applications, it is desirable to detect the presence of pathogens or exogenous nucleic acids and to detect or quantify RNA transcript levels.

In light of the increasing amount of sequence data that is becoming available for various organisms, and particularly for higher organisms such as humans, there is a need for rapid and convenient methods for determining the presence or absence of allelic variants, such as single nucleotide polymorphisms, and target mutations. Ideally, such a method should have high sensitivity, accuracy, and reproducibility. Also, the method should allow simultaneous detection of multiple target sequences in a single reaction mixture.

### SUMMARY

In some embodiments of the invention there is provided a method for detecting at least one target polynucleotide sequence in a sample. A ligation mixture is formed that comprises the sample and a plurality of different ligation probe sets, where each ligation probe set comprises (a) a first ligation probe comprising a first target-specific portion, and (b) a second ligation probe comprising a second target-specific portion. The first or second probe in each set further comprises an identifier tag portion that identifies the probe that contains the identifier tag portion. The first and second target-specific portions of first and second ligation probes from at least one ligation probe set are hybridized to adjacent sequences in a target polynucleotide. The ligation reaction mixture is subjected to at least one cycle of ligation, thereby forming at least one first strand comprising the first target-specific portion, the second target-specific portion, and the identifier tag portion of at least one ligation probe set. Subsequently, a second strand is formed that is complementary to at least one first strand and that comprises sequence portions that are complementary to the first target-specific portion, the second target-specific portion, and the identifier tag of at least one ligation probe set. An optional further amplification of the first strand, the second strand, or both, may then be performed. One or more complexes are subsequently formed, wherein each complex comprises a first strand, a second strand, or both, and a mobility probe. The mobility probe comprises (a) a mobility defining moiety that imparts an identifying mobility or total mass to the mobility probe, and (b) a tag portion or tag portion complement, wherein the tag portion or tag portion complement is hybridized to the complementary tag portion complement or tag portion, respectively, in the first or second strand in one or more complexes. One or more different mobility probes are released from the one or more complexes, and detection of the one or more released mobility probes accomplished using a mobility-dependent analysis technique (MDAT). Additional embodiments of the inventions herein can be found in the appended claims.

In some embodiments, there is provided a method for detecting at least one target polynucleotide sequence in a sample. In the method, a sample that contains, or may contain, a plurality of target sequences is combined with a plurality of different probe sets. Each probe set comprises (a) a first probe comprising a first target-specific portion and a 5′ primer-specific portion, and (b) a second probe comprising a second target-specific portion and a 3′ primer-specific portion, wherein the first and second probes in each set are suitable for ligation together when hybridized to adjacent complementary target sequences. The first or second probe in each set further comprises an identifier tag portion that is between the primer-specific portion and the target-specific portion. The identifier tag portion identifies the probe that contains the identifier tag portion.

The ligation reaction mixture is subjected to at least one cycle of ligation, wherein adjacently hybridized first and second probes of at least one probe set are ligated together to form a ligation product comprising a 5′ primer-specific portion, first and second target-specific portions, a 3′ primer-specific portion, and an identifier tag portion, to form a first strand.

In some embodiments, the first or second probe comprises an affinity moiety, such as biotin, for use in a solid-phase separation step. For example, the second probe can comprise an affinity moiety at its 3′ end, so that the resulting ligation product can be captured by a support-bound affinity partner, such as streptavidin, to allow non-ligation components of the reaction mixture to be washed away. Alternatively, in another non-limiting example, the first probe may comprise an affinity moiety at its 5′ end. In some embodiments, capture is performed prior to ligation. In some embodiments, capture is performed after ligation, before amplification.

In some embodiments, unligated probes can be selectively degraded after ligation by exonuclease treatment to remove unligated probes that are susceptible to exonuclease digestion.

In some embodiments, the first strand from the ligation reaction is combined with a reverse primer that is complementary to the 3′ primer-specific portion, and the primer is extended with a polymerase to form a double-stranded product comprising the first strand and a complementary, second strand that is hybridized to the first strand.

In some embodiments, the first strand, the second strands, or both, are amplified by polymerase-mediated extension of a forward primer and/or the reverse primer, wherein the forward primer is complementary to the complement of the 5′ primer-specific portion, to form amplified first strand and/or second strands.

Subsequent to amplification, one or more complexes are formed, wherein each complex comprises an amplified strand and a mobility probe. The mobility probe comprises (a) a mobility defining moiety that imparts an identifying mobility or total mass to the mobility probe, and (b) a tag portion or tag portion complement, wherein the tag portion or tag portion complement is hybridized to the complementary tag portion complement or tag portion, respectively, in the amplified strand. In some embodiments, for example those in which detection is performed by spectrophotometric methods (e.g., fluorescence detection), the mobility probe may additionally comprise (c) a detectable label, such as a fluorescent label.

In some embodiments, complexes are captured on a solid support. For this purpose, the first probe, second probe, first primer, or second primer may include an affinity moiety (which may be the same or different from the affinity moiety mentioned above in connection with ligation, if used), and the solid support comprises an affinity moiety binding partner. After an amplified strand is captured on the solid support, the support may be washed to remove undesired reaction components. In some embodiments, undesired reaction components may be removed by size exclusion chromatography, ultrafiltration, exonuclease treatment, or other technique, with or without using an affinity capture step.

Following complex formation, and optional affinity capture and washing, one or more mobility probes are released from the one or more complexes and are detected by a mobility-dependent analysis technique (MDAT), such as electrophoresis, chromatography, or mass spectrometry. From the presence or absence of a particular mobility probe, as evidenced by its particular mobility observed by the MDAT, the presence or absence of each target sequence can be determined.

In some embodiments, a probe set is used that comprises a probe that contains a T nucleotide at a selected position to detect conversion of cytosine to uracil (indicating that the cytosine was not methylated), and a second probe that contains an A nucleotide at the selected position to detect conversion of cytosine to thymine (indicating that the cytosine was methylated). The relative amounts of T versus A that are detected can also be used to estimate the average amount of methylation for one or more particular cytosine nucleotides.

In some embodiments, each sample is treated separately during the steps of ligation, amplification, and hybridization of mobility probes. During the samples' ligation reaction, the tag portion of a probe specifies a particular target sequence (for example, allelic variant). By hybridizing mobility probes that possess different mobility modifiers for a given tag portion to the eventual product in the separate pooled samples, eluting the bound mobility probes, and subsequently mixing the pooled samples' mobility probes for MDAT analysis, the relative amounts of target sequence in the pools may be ascertained by ratio calculation.

More broadly, the invention also includes methods as described above, but wherein the amplification step is optional, and the ligation step optionally includes one or more cycles of exponential ligation (using ligation-competent or ligation-incompetent probe sets) to increase the amount of ligation product (and its complement). For such ligase chain reactions, the probe sets comprise third and fourth probes that are complementary to the target-specific portions of the first and second probes, respectively, to generate one or more copies of the complementary strand of the first ligation product. Thus, for such embodiments, primer-specific portions are unnecessary and can be omitted from the probes. Following ligation and optional additional cycles of probe ligation, the ligation products and/or their complements can be combined with mobility probes as above, to form complexes that comprise a ligation product (single or double stranded) and a mobility probe that are hybridized together via the complementary tag portion and tag portion complement. The mobility probes may then be released and detected, to determine the presence or absence of the target sequences. Also provided are reagents and kits that may be useful in practicing various methods of the invention.

These and other features and advantages of the invention will become more readily apparent in light of the description of various embodiments herein.

These and other features and advantages of the invention will become more readily apparent in light of the following figures. The figures are not intended to limit the scope of the present teachings in any way.

### BRIEF DESCRIPTION OF THE FIGURES

FIG. 1 illustrates an exemplary probe set in accordance with some embodiments of the invention.

FIG. 2 illustrates a way to differentiate between two potential alleles in a target locus by ligation, in accordance with certain embodiments of the invention.

FIG. 3 illustrates an exemplary scheme in accordance with some embodiments of the invention.

FIG. 4 illustrates another exemplary scheme in accordance with some embodiments of the invention.

FIG. 5 shows a simplified electropherogram of several mobility probe peaks.

### DESCRIPTION OF VARIOUS EMBODIMENTS

The present invention provides methods for detecting one or more selected target polynucleotide sequences in a sample. The invention permits detection of target sequences with high specificity and sensitivity, allowing detection and/or quantitation of small amounts of target sequences. In some embodiments, the invention is also advantageous for genotyping and detection of genetic polymorphisms.

It is to be understood that both the foregoing general description and the following detailed description are exemplary and explanatory only and are not restrictive of the invention. In this application, the use of the singular includes the plural unless specifically stated otherwise. For example, “a probe” means that more than one probe may be present. Also, the use of “or” means “and/or” unless stated otherwise or necessitated by the context. Similarly, “comprise”, “comprises”, “comprising”, “include”, “includes”, and “including” are not intended to be limiting.

The term “nucleoside” refers to a compound comprising a purine, deazapurine, or pyrimidine nucleobase, e.g., adenine, guanine, cytosine, uracil, thymine, 7-deazaadenine, 7-deazaguanosine, and the like, that is linked to a pentose at the 1′-position. When the nucleoside base is purine or 7-deazapurine, the pentose is attached to the nucleobase at the 9-position of the purine or deazapurine, and when the nucleobase is pyrimidine, the pentose is attached to the nucleobase at the 1-position of the pyrimidine.

The term “nucleotide” as used herein refers to a phosphate ester of a nucleoside, e.g., a triphosphate ester, wherein the most common site of esterification is the hydroxyl group attached to the C-5 position of the pentose. See, e.g., Kornberg and Baker, DNA Replication, 2nd Ed. (Freeman, San Francisco, 1992).

The term “polynucleotide” means polymers of nucleotide monomers, including analogs of such polymers, including double- and single-stranded deoxyribonucleotides, ribonucleotides, alpha and beta-anomeric forms thereof, and the like. Monomers are linked by “internucleotide linkages,” e.g., phosphodiester linkages, where as used herein, the term “phosphodiester linkage” refers to phosphodiester bonds or bonds including phosphate analogs thereof, including associated counterions, e.g., H⁺, NH₄+, Na⁺, if such counterions are present. Whenever a polynucleotide is represented by a sequence of letters, such as “ATGCCTG,” it will be understood that: (i) the nucleotides are in 5′ to 3′ order from left to right unless otherwise noted or it is apparent to the skilled artisan from the context that the converse was intended; and (ii) that “A” denotes deoxyadenosine, “C” denotes deoxycytidine, “G” denotes deoxyguanosine, and “T” denotes deoxythymidine. Descriptions of how to synthesize oligonucleotides can be found, among other places, in U.S. Pat. Nos. 4,373,071; 4,401,796; 4,415,732; 4,458,066; 4,500,707; 4,668,777; 4,973,679; 5,047,524; 5,132,418; 5,153,319; and 5,262,530.

“Analogs” in reference to nucleosides and/or polynucleotides comprise synthetic analogs having modified nucleobase portions, modified pentose portions and/or modified phosphate portions, and, in the case of polynucleotides, modified internucleotide linkages, as described generally elsewhere (e.g., Scheit, Nucleotide Analogs (John Wiley, New York, (1980); Englisch, Angew. Chem. Int. Ed. Engl. 30:613-29 (1991); Agrawal, Protocols for Polynucleotides and Analogs, Humana Press (1994)). Generally, modified phosphate portions comprise analogs of phosphate wherein the phosphorous atom is in the +5 oxidation state and one or more of the oxygen atoms are replaced with a non-oxygen moiety, e.g., sulfur. Exemplary phosphate analogs include but are not limited to phosphorothioate, phosphorodithioate, phosphoroselenoate, phosphorodiselenoate, phosphoroanilothioate, phosphoranilidate, phosphoramidate, boronophosphates, including associated counterions, if such counterions are present. Exemplary modified nucleobase portions include but are not limited to 2,6-diaminopurine, hypoxanthine, pseudouridine, C-5-propyne, isocytosine, isoguanine, 2-thiopyrimidine, and other like analogs. According to some embodiments, nucleobase analogs can include iso-C and iso-G nucleobase analogs available from Sulfonics, Inc., Alachua, Fla. (e.g., Benner, et al., U.S. Pat. No. 5,432,272) or LNA analogs (“Locked Nucleic Acids, e.g., Koshkin et al., Tetrahedron 54:3607-30 (1998), WO 98/39352, WO 99/14226, WO 00/56746, and WO 99/60855). Exemplary modified pentose portions include but are not limited to 2′- or 3′-modifications where the 2′- or 3′-position is hydrogen, hydroxy, alkoxy, e.g., methoxy, ethoxy, allyloxy, isopropoxy, butoxy, isobutoxy and phenoxy, azido, amino or alkylamino, fluoro, chloro, bromo and the like. Modified internucleotide linkages include, but are not limited to, phosphate analogs, analogs having achiral and uncharged intersubunit linkages (e.g., Sterchak, E. P., et al., Organic Chem, 52:4202 (1987)), and uncharged morpholino-based polymers having achiral intersubunit linkages (e.g., U.S. Pat. No. 5,034,506). Internucleotide linkage analogs include, but are not limited to, peptide nucleic acid (PNA), morpholidate, acetal, and polyamide-linked heterocycles. In some embodiments, one may use a class of polynucleotide analogs where a conventional sugar and internucleotide linkage has been replaced with a 2-aminoethylglycine amide backbone polymer is PNA (e.g., Nielsen et. al., Science, 254:1497-1500 (1991); Egholm et al., J. Am. Chem. Soc., 114: 1895-1897 (1992)).

A “target” or “target nucleic acid sequence” according to the present invention comprises a specific nucleic acid sequence that is to be detected (and possibly quantified). The term “target nucleic acid sequence” encompasses both DNA, RNA, and any analog thereof that have the ability to form base-paired duplexes or triplexes. The person of ordinary skill will appreciate that while the target nucleic acid sequence may be described as a single-stranded molecule, the complement of that single-stranded molecule, or a double-stranded target nucleic acid molecule may also serve as a target nucleic acid sequence. In addition, a target nucleic acid sequence may be the actual target nucleic acid present in a sample, or it may be a counterpart of that sequence, such as a cDNA derived from a target RNA sequence present in the starting material. In some embodiments, the target nucleic acid sequence may comprise single- or double-stranded DNA; cDNA, either single-stranded or double-stranded (e.g., DNA:DNA and DNA:RNA hybrids); and RNA, including, but not limited to, mRNA, mRNA precursors, and rRNA.

As used herein, “detecting” encompasses detection, quantification, and/or identification.

The term “amplification product” as used herein refers to the product of an amplification reaction including, but not limited to, primer extension, the polymerase chain reaction, RNA transcription, ligase chain reaction, repair ligase chain reaction, linear amplification embodiments thereof, and the like. Thus, exemplary amplification products may comprise primer extension products, PCR amplicons, ligation amplicons, RNA transcription products, and/or the like.

The target nucleic acids for use with the invention may be derived from any organism or other source, including but not limited to prokaryotes, eukaryotes, plants, animals, and viruses, as well as synthetic nucleic acids, for example. Target nucleic acids may originate from any of a wide variety of sample types, such as cell nuclei (e.g., genomic DNA), whole cells, tissue samples, phage, plasmids, mitrochondria (containing mDNA), and the like. To reduce viscosity or improve hybridization kinetics, target nucleic acids may be sheared prior to use in the invention.

Many methods are available for the isolation and purification of target nucleic acids. Preferably, the target nucleic acids are sufficiently free of proteins and any other interfering substances to allow adequate target-specific probe annealing, cleavage, and ligation. Exemplary purification methods include (i) organic extraction followed by ethanol precipitation, e.g., using a phenol/chloroform organic reagent (Ausubel et al., eds., Current Protocols in Molecular Biology Vol. 1, Chapter 2, Section I, John Wiley & Sons, New York (1993)), preferably with an automated DNA extractor, e.g., a Model 341 DNA Extractor from Applied Biosystems (Foster City, Calif.); (ii) solid phase adsorption methods (Walsh et al., Biotechniques 10(4): 506-513, 1991; Boom et al., U.S. Pat. No. 5,234,809); and (iii) salt-induced DNA precipitation methods (Miller et al., Nucleic Acids Res. 16(3):9-10, 1988), such methods being typically referred to as “salting-out” methods. Optimally, each of the above purification methods is preceded by an enzyme digestion step to help eliminate protein from the sample, e.g., digestion with proteinase K, or other proteases.

To facilitate detection, the target nucleic acid can be amplified using a suitable amplification procedure prior to ligation and (optional) amplification steps of various embodiments of the invention. Such amplification may be linear or exponential. In one embodiment, amplification of the target nucleic acid is accomplished using the polymerase chain reaction (PCR) (e.g., Mullis et al., eds, The Polymerase Chain Reaction, BirkHauser, Boston, Mass., 1994). Generally, the PCR consists of an initial denaturation step which separates the strands of a double stranded nucleic acid sample, followed by repetition of (i) an annealing step, which allows amplification primers to anneal specifically to positions flanking a target sequence; (ii) an extension step which extends the primers in a 5′ to 3′ direction thereby forming an amplicon nucleic acid complementary to the target sequence, and (iii) a denaturation step which causes the separation of the amplicon from the target sequence. Each of the above steps may be conducted at a different temperature, preferably using an automated thermocycler (Applied Biosystems, Foster City, Calif.).

If desired, RNA samples can be converted to DNA/RNA heteroduplexes or to duplex cDNA by known methods (e.g., Ausubel et al., supra; and Sambrook et al., Molecular Cloning: A Laboratory Manual, 2nd Edition, Cold Spring Harbor Laboratory, New York (1989); Sambrook and Russell, Molecular Cloning, Third Edition, Cold Spring Harbor Press (2000)). In addition, preparation of target nucleic acids can be accomplished using whole genome amplification techniques (e.g., Lizardi, U.S. Pat. No. 6,124,120).

In some embodiments, target nucleic acids are chemically treated prior to analysis. For example, analysis of the methylation state of cytosines can be performed using bisulfite as a modifying agent (e.g., see U.S. Pat. Nos. 6,265,171 and 6,331,393). Incubating target nucleic acid sequence with bisulfate results in deamination of a substantial portion of unmethylated cytosines, which converts such cytosines to uracil. Methylated cytosines are deaminated to a measurably lesser extent. In some embodiments, the sample is then amplified or replicated, resulting in the uracil bases being replaced with thymine. Thus, in some embodiments, a substantial portion of unmethylated target cytosines ultimately become thymines, while a substantial portion of methylated cytosines remain cytosines. In some embodiments, the identity of the nucleotide (cytosine, uracil, or thymine) of the target may be determined by a ligation and amplification method of the present invention, wherein a probe set is designed to detect the presence of either uracil or thymine at a known cytosine position in a bisulfite-treated target nucleic acid. In some embodiments, a probe set is used that comprises a probe that contains a T nucleotide at a selected position to detect conversion of cytosine to uracil (indicating that the cytosine was not methylated), and a second probe that contains an A nucleotide at the selected position to detect conversion of cytosine to thymine (indicating that the cytosine was methylated). The relative amounts of T versus A that are detected can also be used to estimate the average amount of methylation for one or more particular cytosine nucleotides.

The present invention employs probes that are designed to hybridize to complementary target sequences, and which are capable of undergoing ligation when hybridized to adjacent complementary regions in a target sequence. In some embodiments, probes of the invention can be used, for example, in linear and/or exponential probe ligation methods described herein. In some embodiments, ligation probes are designed to be initially ligation-incompetent, such that ligation does not occur until one or more probes are modified to a ligation-competent form. For example, a ligation probe can be designed to contain a blocking group or non-complementary polynucleotide portion that prevents ligation with another ligation probe, as discussed further below.

Different probe sets can be prepared, wherein each probe set comprises at least a first probe and a second probe.

In some embodiments, a plurality of different ligation probe sets are mixed with a sample, such that each ligation probe set comprising (a) a first ligation probe comprising a first target-specific portion, and (b) a second ligation probe comprising a second target-specific portion, wherein the first or second probe in each set further comprises an identifier tag portion that identifies the probe that contains the identifier tag portion. In some embodiments, the first and second target-specific portions of first and second ligation probes from at least one ligation probe set can hybridize to adjacent sequences in a target polynucleotide.

In some embodiments, at least one probe set is designed to be ligation-incompetent. However, the probe set can be converted to a ligation-competent form to allow ligation, as discussed below.

In some embodiments, the first probe comprises a first target-specific portion and a 5′ primer-specific portion, and the second probe comprises a second target-specific portion and a 3′ primer-specific portion. The first probe and the second probe in each set are designed to be suitable for ligation of the first target-specific portion to the second target specific portion when the first and second target-specific portions are hybridized to adjacent complementary target sequences, although, as mentioned above probes may be initially ligation-incompetent if desired. For example, the first probe can be designed such that the first target-specific portion is located on the 3′ end of the first probe, and the second probe can be designed so that the second target-specific portion is located on the 5′ end of the second probe. When the first and second probe are hybridized to adjacent complementary regions in the target sequence (adjacent target regions), the 3′ end of the first probe can be ligated to the 5′ end of the second probe.

The length of the target-specific portion in each probe is selected to ensure specific hybridization of the probe to the desired target sequence, without significant cross-hybridization to non-target nucleic acids. Also, to enhance binding specificity, the melting temperatures of the target-specific portions can be selected to be within a few degrees of each other. In some embodiments, the melting temperatures (Tm) of the target-specific portions are within a ΔTm range (Tmax-Tmin) of 10° C. or less, 5° C. or less, 3° C. or less, or 2° C. or less. This can be accomplished by suitable choice of sequence lengths for target-specific portions based on known methods for predicting melting temperatures (Breslauer et al., Proc. Natl. Acad. Sci. 83:3746-3750 (1986); Rychlik et al., Nucleic Acids Res. 17:8543-8551 (1989) and 18:6409-6412 (1990); Wetmur, Crit. Rev. Biochem. Mol. Biol 26:227-259 (1991); Osborne, CABIOS 8:83 (1991); Montpetit et al., J. Virol. Methods 36:119-128 (1992); and Kwok et al., Nucl. Acid Res. 18:999-1005, 1990), for example. See also Zuker et al, Algorithms and Thermodynamics for RNA Secondary Structure Prediction: A Practical Guide, in RNA Biochemistry and Biotechnology, pages 11-43, J. Barciszewski & B. F. C. Clark, eds., NATO ASI Series, Kluwer Academic Publishers (1999). Also, Version 3.0 of mfold for Unix operating systems is available via a free license for academic and nonprofit use only; commercial use is available for a fee. Copyright© is held by Washington University.) Target-specific portions having lengths from 12 to 35 bases, 15 to 30 bases, or from 16 to 24 bases, for example, tend to be very sequence-specific when the annealing temperature is set within a few degrees of a probe melting temperature (Dieffenbach et al., in PCR Primer: A Laboratory Manual, Dieffenbach and Dveksler, eds., pp. 133-142, CSHL Press, New York (1995)). However, longer or shorter sequences can also be used. Also, when nucleotide analogs that have higher binding affinities for complementary nucleotides are included in a probe sequence (e.g., locked-nucleic acids, also called “LNAs”; see above), a shorter probe sequence can be used to achieve a particular Tm or particular target specificity.

In some embodiments, a primer-specific portion can be included in ligation probes to facilitate amplification of the ligation product (one or both of the sense strand and the antisense strand) by allowing hybridization of a complementary primer for primer extension. The 3′ primer-specific portion, which is located downstream of (3′ relative to) the second target-specific portion in the second probe, can serve as a template for hybridizing to a complementary primer (the “second primer”), followed by primer extension to form a second strand that is complementary to the first strand that was formed by ligation of the first and second probes. Extension of the second primer through the 5′ primer-specific portion in the first strand creates a complement of the 5′ primer specific portion, which can serve as a template for hybridizing to a complementary primer (the “first primer”). Primer extension of this first primer can be used to generate a new copy of the first strand.

In some embodiments, ligation probes do not contain primer-specific portions, and primer extension is unnecessary.

In some embodiments, the primer-specific portions in each probe set (and thus, the first and second primers that are complementary to the primer-specific portions) are designed to have Tm values that are within a ΔTm range of 10° C. or less, 5° C. or less, 3° C. or less, or 2° C. or less. In some embodiments, the first and second primer-specific portions in a plurality of probe sets are designed to have Tm values that are within a ΔTm range of 10° C. or less, 5° C. or less, 3° C. or less, or 2° C. or less. In some embodiments, the first primer-specific portions in first probes from a plurality of the probe sets are identical to each other, so that a plurality of different ligation products (first strands generated by ligation of first and second probes from different probe sets) can be amplified simultaneously by extending the same first complementary primer. In some embodiments, the second primer-specific portions in second probes from a plurality of the probe sets are identical to each other, so that a plurality of different second strands (generated by forming the complement of the first strand by extending the second primer) can be amplified simultaneously by extending the same second primer. Tm values can be calculated for such primer-specific portions using the references cited above for the target-specific portions.

A “universal primer” is capable of hybridizing to the primer-specific portion of first or second probes from more than one probe set, ligation product, or amplification product, as appropriate. A “universal primer set” comprises a first primer and a second primer that hybridize with a plurality or selected subset of species of probes, ligation products, or amplification products, as appropriate. In some embodiments, the universal primer or the universal primer set hybridizes with all or most of the probes, ligation products, or amplification products in a reaction, as appropriate. When universal primer sets are used in some amplification reactions, such as, but not limited to, PCR, quantitative results may be obtained for a broad range of template concentrations.

In some embodiments, universal primer sequences of both upstream and downstream ligation probes can be designed such that a single primer can be used in a subsequent amplification reaction.

In some embodiments, primers or primer-specific portions may include additional guanosine and cytosine in order to increase the stringency of hybridization per unit length, thereby shortening the overall primer sequence.

In some embodiments, universal primers can include sequence modifications to potentially achieve better performance, according to the needs of the user. In some embodiments, universal primers are designed to reduce formation of primer dimers, for example, by including regions of sequence identity at the 5′ ends of both the forward and reverse primers to encourage hairpin formation of primer dimers, removing them from the pool of amplifiable species. In some embodiments, universal primers are modified to minimize heteroduplex formation of amplification products, for example by introducing sequence degeneracy to reduce the complementary of the amplified ends of different amplicons.

The first or second probe in each set comprises an identifier tag portion that is distal to the end to the terminus of the target-specific portion that is to be ligated to the other probe, and is between the target-specific portion and the primer-specific portion when the primer-specific portion is present. The identifier tag portion can be used to identify the probe that contains the identifier tag portion, as explained further below. Thus, the tag sequences should be selected to minimize (1) internal, self-hybridization, (2) hybridization with other same-sequence tags, (3) hybridization with other, different sequence tag complements, (4) and hybridization with the sample polynucleotides. Similar considerations apply to the target-specific portions and the primer-specific portions as well. Also, in some embodiments each identifier tag portion can specifically recognize and hybridize to its corresponding tag portion complement under the same conditions for all tags.

Sequences of identifier tag portions can be selected by any suitable method. For example, computer algorithms for selected non-cross hybridizing sets of tags are described in Brenner (PCT Publications No. WO 96/12014 and WO 96/41011) and Shoemaker (Shoemaker et al., European Pub. No. EP 799897 A1 (1997)). In some embodiments, the tag portions have Tm values that are within a preselected temperature range, as discussed above with respect to the target-specific portions or primer-specific portions. In some embodiments, the melting temperatures of the tag portions are within a ΔTm range of 110° C. or less, 5° C. or less, 3° C. or less, or 2° C. or less. In some embodiments, the tag portions in a plurality or all of the probe sets are designed to have Tm values that are within a ΔTm range of 10° C. or less, 5° C. or less, 3° C. or less, or 2° C. or less. In some embodiments, the tag segments are at least 12 bases in length to facilitate specific hybridization to corresponding tag complements. In some embodiments, tag segments are from 12 to 60 bases in length, or 15 to 30 bases in length.

The lengths of target-specific portions, primers, primer segments, tag segments, and/or tag complements can be significantly shortened (e.g., to 12 to 14 nt) by including nucleotide analogs having higher binding affinities than standard nucleotides for complementary nucleotides, such as LNAs or PNAs. In some embodiments, LNAs and/or PNAs nucleotides are included in a proportion of up to 40 or 50% of the total number of nucleotides, optionally in an alternating manner, separated by standard nucleotides. However, higher proportions of LNAs and/or PNAs can also be used. In some embodiments, cytosine and thymine LNA bases are advantageous.

In some embodiments, the first and second probes of at least one different probe set are provided in a covalently linked form, such that the first probe is covalently linked by its 5′ end to the 3′ end of the second probe by a linking moiety. In one embodiment, the linking moiety comprises a chain of polynucleotides that are not significantly complementary to the target strand, the probes, or to any other nucleic acid in the sample. The linking moiety is sufficiently long to allow the target-complementary sequences in the probes to hybridize to the target strand region and to form a viable hybridization complex for cleavage. In some embodiments, the linking moiety is at least 10 nucleotides longer than the collective length of the first and second target regions. A polynucleotide linking moiety can contain or consist of any suitable sequence. For example, the linking moiety can be a homopolymer of C, T, G or A. Alternatively, the linking moiety can contain or consist of a non-nucleotidic polymer, such as polyethylene glycol, a polypeptide such as polyglycine, etc.

In some embodiments, at least one primer set comprises at least one first primer. The first primer of a primer set is designed to hybridize with the complement of the 5′ primer-specific portion of that same ligation or amplification product in a sequence-specific manner. According to some embodiments, a primer set of the present invention comprises at least one second primer. The second primer in that primer set is designed to hybridize with a 3′ primer-specific portion of a ligation or amplification product in a sequence-specific manner. In some embodiments, at least one primer of the primer set comprises a promoter sequence or its complement or a portion of a promoter sequence or its complement. For a discussion of primers comprising promoter sequences, see Sambrook and Russell.

According to some embodiments, some probe sets may comprise more than one first probe or more than one second probe to allow sequence discrimination between target sequences that differ by one or more nucleotides.

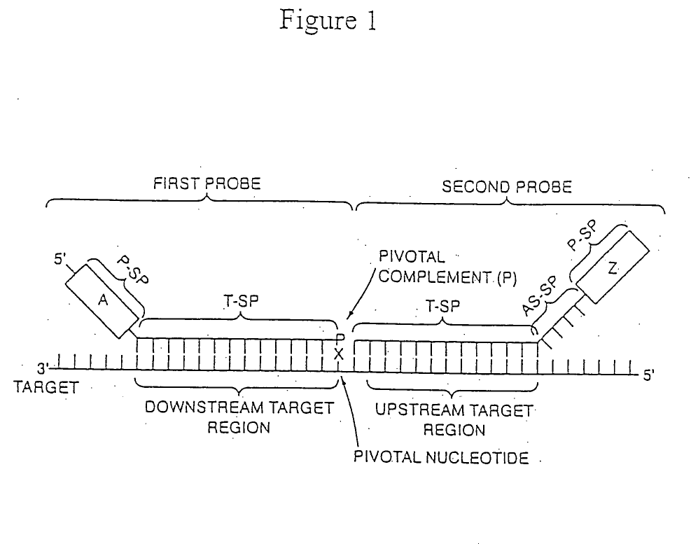

According to some embodiments of the invention, a target-specific probe set can be designed so that the target-specific portion of the first probe will hybridize with the downstream target region (see, e.g., probe A in FIG. 1) and the target-specific portion of the second probe will hybridize with the upstream target region (see, e.g., probe Z in FIG. 1). A nucleotide base complementary to the pivotal nucleotide, the “pivotal complement,” is present on the proximal end of either the first probe (3′ end) or the second probe (5′ end) of the target-specific probe set.

When the first and second probes of the probe set are hybridized to the appropriate upstream and downstream target regions, and the pivotal complement is base-paired with the pivotal nucleotide on the target sequence, the hybridized first and second probes may be ligated together to form a ligation product (see, e.g., FIG. 1(B)-(C)). A mismatched base at the pivotal nucleotide, however, impedes ligation, even if both probes are otherwise fully hybridized to their respective target regions. Thus, highly related sequences that differ by as little as a single nucleotide can be distinguished.

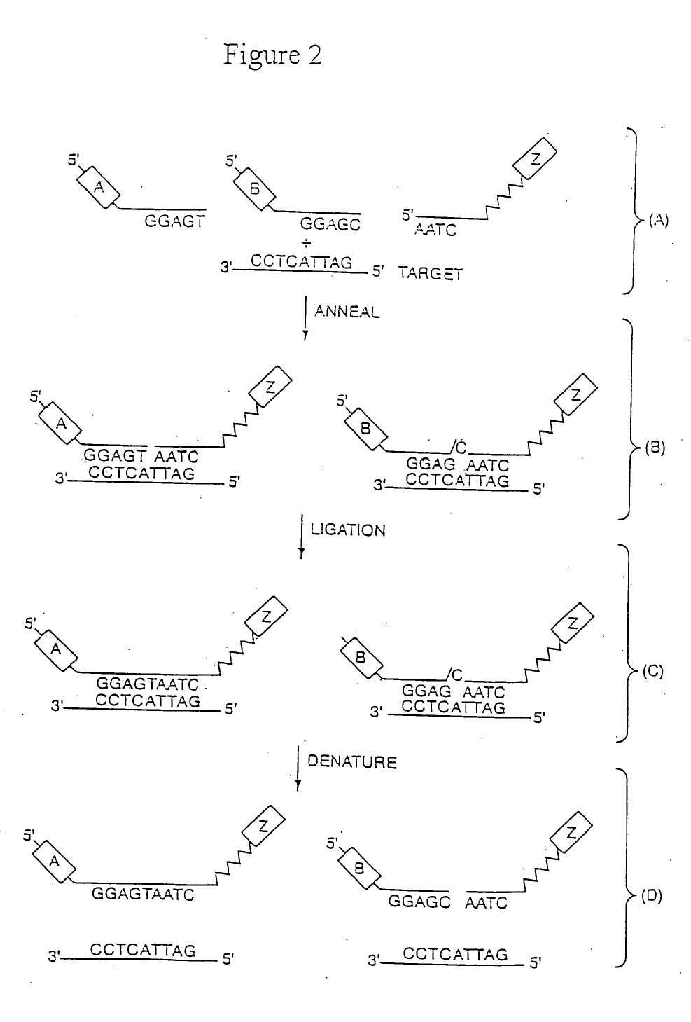

For example, according to some embodiments, one can distinguish the two potential alleles in a biallelic locus as follows. A probe set comprising two first probes, differing in their primer-specific portions and their pivotal complement (see, e.g., probes A and B in FIG. 2(A)) is combined with a second probe (see, e.g., probe Z in FIG. 2(A)) and a sample containing target nucleic acids. All three probes will hybridize with the target sequence under appropriate conditions (see, e.g., FIG. 2(B)). Only the first probe with the hybridized pivotal complement, however, will be ligated with the hybridized second probe (see, e.g., FIG. 2(C)). Thus, if only one allele is present in the sample, only one ligation product for that target will be generated (see, e.g., ligation product A-Z in FIG. 2(D)). Both ligation products would be formed from a heterozygous sample.

Further, in some embodiments, probe sets do not comprise a pivotal complement at the terminus of the first or the second probe. Rather, the target nucleotide or nucleotides to be detected are located within either the 3′ or 5′ target region to which the first probe or second probe hybridizes. Probes with target-specific portions that are fully complementary with their respective target regions can hybridize under stringent conditions. Probes with one or more mismatched bases in the target-specific portion, by contrast, will not hybridize to their respective target region. Both the first probe and the second probe must be hybridized to the target for a ligation product to be generated. Thus, nucleotides to be detected may be pivotal or internal or both.

In some embodiments, the first probes and second probes in a probe set are designed with similar melting temperatures (Tm). Where a probe includes a pivotal complement, the Tm for the probe(s) comprising the pivotal complement(s) of the target pivotal nucleotide can be designed to be approximately 4-6° C. lower than the Tm values of the other probe(s) that do not contain the pivotal complement in the probe set. The probe comprising the pivotal complement(s) will also preferably be designed with a Tm near the ligation temperature. Thus, in these exemplary embodiments, a probe with a mismatched nucleotide will more readily dissociate from the target at the ligation temperature. Thus, the ligation temperature can provide another way to discriminate between, for example, multiple potential alleles in the target.

A ligation agent according to the present invention may comprise any number of enzymatic or chemical (i.e., non-enzymatic) agents. For example, ligase is an enzymatic ligation agent that, under appropriate conditions, forms phosphodiester bonds between the 3′-OH and the 5′-phosphate of adjacent polynucleotides. Temperature-sensitive ligases, include, but are not limited to, bacteriophage T4 ligase, bacteriophage T7 ligase, and E. coli ligase. Thermostable ligases include, but are not limited to, Taq ligase, Tth ligase, and Pfu ligase. Thermostable ligase may be obtained from thermophilic or hyperthermophilic organisms, including but not limited to, prokaryotic, eucaryotic, or archael organisms. Some RNA ligases may also be employed in the methods of the invention.

Chemical ligation agents include, without limitation, activating, condensing, and reducing agents, such as carbodiimide, cyanogen bromide (BrCN), N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of the invention. Detailed protocols for chemical ligation methods and descriptions of appropriate reactive groups can be found, among other places, in Xu et al., Nucleic Acid Res., 27:875-81 (1999); Gryaznov and Letsinger, Nucleic Acid Res. 21:1403-08 (1993); Gryaznov et al., Nucleic Acid Res. 22:2366-69 (1994); Kanaya and Yanagawa, Biochemistry 25:7423-30 (1986); Luebke and Dervan, Nucleic Acids Res. 20:3005-09 (1992); Sievers and von Kiedrowski, Nature 369:221-24 (1994); Liu and Taylor, Nucleic Acids Res. 26:3300-04 (1999); Wang and Kool, Nucleic Acids Res. 22:2326-33 (1994); Purmal et al., Nucleic Acids Res. 20:3713-19 (1992); Ashley and Kushlan, Biochemistry 30:2927-33 (1991); Chu and Orgel, Nucleic Acids Res. 16:3671-91 (1988); Sokolova et al., FEBS Letters 232:153-55 (1988); Naylor and Gilham, Biochemistry 5:2722-28 (1966); U.S. Pat. No. 5,476,930; and Royer, EP 324616B1). In some embodiments, the ligation agent is an “activating” or reducing agent. It will be appreciated that if chemical ligation is used, the 3′ end of the first probe and the 5′ end of the second probe should include appropriate reactive groups to facilitate the ligation.

In some embodiments, for strand synthesis and or amplification, a polymerase is used. In some embodiments, the polymerase may comprise at least one thermostable polymerase, including, but not limited to, Taq, Pfu, Vent, Deep Vent, Pwo, UITma, and Tth polymerase and enzymatically active mutants and variants thereof. Such polymerases are well known and/or are commercially available. Descriptions of polymerases can be found, among other places, at the world wide web URL: the-scientist.library.upenn.edu/yr1998/jan/profile 1_{—}980105.html.

The present teachings also employ mobility probes that are useful for detecting amplified ligation products using a mobility- or mass-dependent analysis technique. Each mobility probe comprises (a) a mobility defining moiety that imparts an identifying mobility or total mass to the mobility probe, (b) a tag portion or tag portion complement for hybridizing to a complementary tag portion complement or tag portion, respectively, in an amplified strand. For each different target sequence to be detected (e.g., for a different locus or for a particular SNP), a different mobility probe is used which has a distinct tag portion or tag portion complement and a distinct mobility defining moiety which allows the attached tag portion or tag portion complement (and the corresponding target sequence) to be identified from the distinct mobility or total mass of the mobility probe. Mobility probes may also comprise (c) a label.

In the following discussion and in the figures, the construct comprising the mobility-defining moiety, the tag portion, and the optional label, can be referred to as a mobility probe (MP). Further, any of a variety of different mobility probe constructs and configurations can be used. In the following discussion, although the mobility defining moiety may be referred to as a “tail” or “tail portion”, such wording is not intended to limit the structure of the mobility defining moiety.

The tail portion of a mobility defining moiety may be any entity capable of achieving a particular mobility or total mass. In certain embodiments, the mobility defining moiety should (1) have a low polydispersity in order to effect a well-defined and easily resolved mobility, e.g., Mw/Mn less than 1.05; (2) be soluble in an aqueous medium; (3) not adversely affect probe-target hybridization; and (4) be available in sufficient number such that mobility probes for different probe sets have distinguishable mobilities or total masses.

In some embodiments, the tail portion comprises a polymer. For example, the polymer may be homopolymer, random copolymer, or block copolymer. Furthermore, the polymer may have a linear, comb, branched, or dendritic structure. In addition, although the invention is described herein with respect to a single polymer chain attached to an associated mobility defining moiety, the invention also contemplates mobility defining moieties comprising more than one polymer chain element, where the elements collectively form a tail portion.

Exemplary polymers for use in the present invention include, but are not limited to, hydrophilic, or at least sufficiently hydrophilic when bound to a tag complement to ensure that the tag complement is readily soluble in aqueous medium. Where the mobility-dependent analysis technique is electrophoresis, the polymers can be designed for some embodiments of the invention to be uncharged or have a charge/subunit density that is substantially less than that of the amplification product.

In certain embodiments, the polymer comprises polyethylene oxide (PEO), e.g., formed from one or more hexaethylene oxide (HEO) units, where the HEO units are joined end-to-end to form an unbroken chain of ethylene oxide subunits. Other exemplary embodiments include a chain composed of n 12mer PEO units, and a chain composed of n tetrapeptide units, where n is an adjustable integer (e.g., Grossman et al., U.S. Pat. No. 5,777,096).

In certain embodiments, the synthesis of polymers useful as tail portions may depend on the nature of the polymer. Methods for preparing suitable polymers generally follow well known polymer subunit synthesis methods. Methods of forming selected-length PEO chains are discussed below. These methods, which involve coupling of defined-size, multi-subunit polymer units to one another, either directly or through charged or uncharged linking groups, are generally applicable to a wide variety of polymers, such as polyethylene oxide, polyglycolic acid, polylactic acid, polyurethane polymers, polypeptides, and oligosaccharides. Such methods of polymer unit coupling are also suitable for synthesizing selected-length copolymers, e.g., copolymers of polyethylene oxide units alternating with polypropylene units. Polypeptides of selected lengths and amino acid composition, either homopolymer or mixed polymer, can be synthesized by standard solid-phase methods (e.g., Fields and Noble, Int. J. Peptide Protein Res., 35: 161-214 (1990)).

In some methods for preparing PEO polymer chains having a selected number of HEO units, an HEO unit is protected at one end with dimethoxytrityl (DMT), and activated at its other end with methane sulfonate. The activated HEO is then reacted with a second DMT-protected HEO group to form a DMT-protected HEO dimer. This unit-addition is then carried out successively until a desired PEO chain length is achieved (e.g., Levenson et al., U.S. Pat. No. 4,914,210).

Another exemplary polymer for use as a tag portion complement is L-DNA. L-DNA polymers can be prepared by standard oligonucleotide synthesis as described above, form the corresponding L-DNA monomers, which are commercially available. One advantage of L-DNA polymers is that they do not hybridize to standard D-DNA polymers, so cross-hybridization problems are reduced.

Coupling of the polymer tails to a polynucleotide tag complement can be carried out by an extension of conventional phosphoramidite polynucleotide synthesis methods, or by other standard coupling methods, e.g., a bis-urethane tolyl-linked polymer chain may be linked to a polynucleotide on a solid support via a phosphoramidite coupling. Alternatively, the polymer chain can be built up on a polynucleotide (or other tag portion) by stepwise addition of polymer-chain units to the polynucleotide, e.g., using standard solid-phase polymer synthesis methods.

The contribution of a tail to the mobility of the mobility probe, in some embodiments, will generally depend on the size of the tail. However, addition of charged groups to the tail, e.g., charged linking groups in the PEO chain, or charged amino acids in a polypeptide chain, can also be used to achieve selected mobility or mass characteristics.

Additional guidance for selection and synthesis of mobility defining moieties can be found in PCT Publications No. WO 00/55368 (Grossman), WO 01/49790 (Menchen et al.), and WO 02/83954 (Woo et al, application No. PCT/US02/11824).

When a tag portion or tag portion complement is a polynucleotide, the tag complement may comprise all, part, or none of the tail portion of the mobility defining moiety. In some embodiments of the invention, the tag portion or tag portion complement may contain part of the tail portion (or vice versa). In some embodiments, the tag portion or tag portion complement does not comprise any portion of the tail portion of the mobility defining moiety. For example, because PNA is uncharged, particularly when using free solution electrophoresis as the mobility-dependent analysis technique, the same PNA oligomer may act as both a tag portion complement and a tail portion of a mobility defining moiety. One advantage of including PNA in the tag portion complement is that it is uncharged, so that the mobility of the mobility probe is reduced relative to the same probe containing DNA instead of PNA.

In some embodiments, the mobility probe may include a hybridization enhancer, where, as used herein, the term “hybridization enhancer” means moieties that serve to enhance, stabilize, or otherwise positively influence hybridization between two polynucleotides, e.g. intercalators (e.g., U.S. Pat. No. 4,835,263), minor-groove binders (e.g., U.S. Pat. No. 5,801,155), and cross-linking functional groups. In some embodiments, the hybridization enhancer is covalently attached to the mobility defining moiety. In some embodiments, a hybridization enhancer for use in the present invention is a minor-groove binder, e.g., netropsin, distamycin, or the like.

In some embodiments, the mobility probes may comprise a detectable label to facilitate detection of the mobility probe, such as a fluorescent moiety. The skilled artisan will appreciate that many such labels are known in the art, such as fluorophores, radioisotopes, chromogens, enzymes, antigens, heavy metals, dyes, magnetic probes, phosphorescence groups, chemiluminescent groups, and electrochemical detection moieties. Exemplary fluorophores include, but are not limited to, rhodamine, cyanine 3 (Cy 3), cyanine 5 (Cy 5), fluorescein, VIC™, NED™, LIZ™, TAMRA™, 5-FAM™, 6-FAM™, and Texas Red (Molecular Probes). (VIC™, NED™, LIZ™, TAMRA™, 5-FAM™, AND 6-FAM™ are all available from Applied Biosystems, Foster City, Calif.) Exemplary radioisotopes include, but are not limited to, ³²P, ³³P, and ³⁵S. Reporter groups also include elements of multi-element indirect reporter systems, e.g., biotin/avidin, antibody/antigen, ligand/receptor, enzyme/substrate, and the like, in which the element interacts with other elements of the system in order to effect a detectable signal. One exemplary multi-element reporter system includes a biotin reporter group attached to a primer and an avidin conjugated with a fluorescent label. Detailed protocols for methods of attaching detectable labels to oligonucleotides and polynucleotides can be found in, among other places, G. T. Hermanson, Bioconjugate Techniques, Academic Press, San Diego, Calif. (1996) and S. L. Beaucage et al., Current Protocols in Nucleic Acid Chemistry, John Wiley & Sons, New York, N.Y. (2000).

In some embodiments, the label comprises a fluorescent moiety (also called a “fluorescent dye”) that comprises a resonance-delocalized system or aromatic ring system that absorbs light at a first wavelength and emits fluorescent light at a second wavelength in response to the absorption event. A wide variety of such dye molecules are known in the art. For example, fluorescent dyes can be selected from any of a variety of classes of fluorescent compounds, such as xanthenes, rhodamines, fluoresceins, cyanines, phthalocyanines, squaraines, and bodipy dyes.

In one embodiment, the dye comprises a xanthene-type dye, which contains a fused three-ring system of the form: 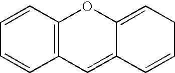
This parent xanthene ring may be unsubstituted (i.e., all substituents are H) or may be substituted with one or more of a variety of the same or different substituents, such as described below.

In one embodiment, the dye contains a parent xanthene ring having the general structure: 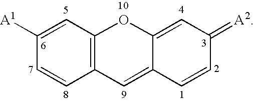
In the parent xanthene ring depicted above, A¹ is OH or NH₂ and A² is O or NH₂+. When A¹ is OH and A² is O, the parent xanthene ring is a fluorescein-type xanthene ring. When A¹ is NH₂ and A² is NH₂+, the parent xanthene ring is a rhodamine-type xanthene ring. When A¹ is NH₂ and A² is O, the parent xanthene ring is a rhodol-type xanthene ring. In the parent xanthene ring depicted above, one or both nitrogens of A¹ and A² (when present) and/or one or more of the carbon atoms at positions C1, C2, C4, C5, C7, C8 and C9 can be independently substituted with a wide variety of the same or different substituents. In one embodiment, typical substituents include, but are not limited to, —X, —R, —OR, —SR, —NRR, perhalo (C₁-C₆) alkyl, —CX₃, —CF₃, —CN, —OCN, —SCN, —NCO, —NCS, —NO, —NO₂, —N₃, —S(O)₂O⁻, —S(O)₂OH, —S(O)₂R, —C(O)R, —C(O)X, —C(S)R, —C(S)X, —C(O)OR, —C(O)O⁻, —C(S)OR, —C(O)SR, —C(S)SR, —C(O)NRR, —C(S)NRR and —C(NR)NRR, where each X is independently a halogen (preferably —F or Cl) and each R is independently hydrogen, (C₁-C₆) alkyl, (C₁-C₆) alkanyl, (C₁-C₆) alkenyl, (C₁-C₆) alkynyl, (C₅-C₂₀) aryl, (C₆-C₂₆) arylalkyl, (C₅-C₂₀) arylaryl, heteroaryl, 6-26 membered heteroarylalkyl 5-20 membered heteroaryl-heteroaryl, carboxyl, acetyl, sulfonyl, sulfinyl, sulfone, phosphate, or phosphonate. Moreover, the C1 and C2 substituents and/or the C7 and C8 substituents can be taken together to form substituted or unsubstituted buta[1,3]dieno or (C₅-C₂₀) aryleno bridges. Generally, substituents that do not tend to quench the fluorescence of the parent xanthene ring are preferred, but in some embodiments quenching substituents may be desirable. Substituents that tend to quench fluorescence of parent xanthene rings are electron-withdrawing groups, such as —NO₂, —Br, and —I. In one embodiment, C9 is unsubstituted. In some embodiments, C9 is substituted with a phenyl group. In some embodiments, C9 is substituted with a substituent other than phenyl.

When A¹ is NH₂ and/or A² is NH₂+, these nitrogens can be included in one or more bridges involving the same nitrogen atom or adjacent carbon atoms, e.g., (C₁-C₁₂) alkyldiyl, (C₁-C₁₂) alkyleno, 2-12 membered heteroalkyldiyl and/or 2-12 membered heteroalkyleno bridges.

Any of the substituents on carbons C1, C2, C4, C5, C7, C8, C9 and/or nitrogen atoms at C3 and/or C6 (when present) can be further substituted with one or more of the same or different substituents, which are typically selected from —X, —R′, ═O, —OR′, —SR′, ═S, —NR′R′, ═NR′, —CX₃, —CN, —OCN, —SCN, —NCO, —NCS, —NO, —NO₂, ═N₂, —N₃, —NHOH, —S(O)₂O⁻, —S(O)₂OH, —S(O)₂R′, —P(O)(O⁻)₂, —P(O)(OH)₂, —C(O)R′, —C(O)X, —C(S)R′, —C(S)X, —C(O)OR′, —C(O)O⁻, —C(S)OR′, —C(O)SR′, —C(S)SR′, —C(O)NR′R′, —C(S)NR′R′ and —C(NR)NR′R′, where each X is independently a halogen (preferably —F or —Cl) and each R′ is independently hydrogen, (C₁-C₆) alkyl, 2-6 membered heteroalkyl, (C₅-C₁₄) aryl or heteroaryl, carboxyl, acetyl, sulfonyl, sulfinyl, sulfone, phosphate, or phosphonate.

Exemplary parent xanthene rings include, but are not limited to, rhodamine-type parent xanthene rings and fluorescein-type parent xanthene rings.

In one embodiment, the dye contains a rhodamine-type xanthene dye that includes the following ring system: 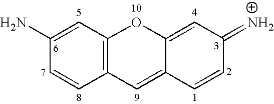
In the rhodamine-type xanthene ring depicted above, one or both nitrogens and/or one or more of the carbons at positions C1, C2, C4, C5, C7 or C8 can be independently substituted with a wide variety of the same or different substituents, as described above for the parent xanthene rings, for example. C9 may be substituted with hydrogen or other substituent, such as an orthocarboxyphenyl or ortho(sulfonic acid)phenyl group. Exemplary rhodamine-type xanthene dyes include, but are not limited to, the xanthene rings of the rhodamine dyes described in U.S. Pat. Nos. 5,936,087, 5,750,409, 5,366,860, 5,231,191, 5,840,999, 5,847,162, and 6,080,852 (Lee et al.), PCT Publications WO 97/36960 and WO 99/27020, Sauer et al., J. Fluorescence 5(3):247-261 (1995), Arden-Jacob, Neue Lanwellige Xanthen-Farbstoffe für Fluoreszenzsonden und Farbstoff Laser, Verlag Shaker, Germany (1993), and Lee et al., Nucl. Acids Res. 20:2471-2483 (1992). Also included within the definition of “rhodamine-type xanthene ring” are the extended-conjugation xanthene rings of the extended rhodamine dyes described in U.S. Pat. No. 6,248,884.

In some embodiments, the dye comprises a fluorescein-type parent xanthene ring having the structure:

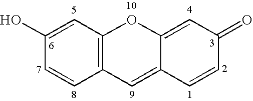

In the fluorescein-type parent xanthene ring depicted above, one or more of the carbons at positions C1, C2, C4, C5, C7, C8 and C9 can be independently substituted with a wide variety of the same or different substituents, as described above for the parent xanthene rings. C9 may be substituted with hydrogen or other substituent, such as an orthocarboxyphenyl or ortho(sulfonic acid)phenyl group. Exemplary fluorescein-type parent xanthene rings include, but are not limited to, the xanthene rings of the fluorescein dyes described in U.S. Pat. Nos. 4,439,356, 4,481,136, 4,933,471 (Lee), U.S. Pat. No. 5,066,580 (Lee), U.S. Pat. Nos. 5,188,934, 5,654,442, and 5,840,999, WO 99/16832, and EP 050684. Also included within the definition of “fluorescein-type parent xanthene ring” are the extended xanthene rings of the fluorescein dyes described in U.S. Pat. Nos. 5,750,409 and 5,066,580.

In some embodiments, the dye comprises a rhodamine dye, which comprises a rhodamine-type xanthene ring in which the C9 carbon atom is substituted with an orthocarboxy phenyl substituent (pendent phenyl group). Such compounds are also referred to herein as orthocarboxyfluoresceins. A particularly preferred subset of rhodamine dyes are 4,7,-dichlororhodamines. Typical rhodamine dyes include, but are not limited to, rhodamine B, 5-carboxyrhodamine, rhodamine X (ROX), 4,7-dichlororhodamine X (dROX), rhodamine 6G (R6G), 4,7-dichlororhodamine 6G (dR6G), rhodamine 110 (R110), 4,7-dichlororhodamine 110 (dR110), tetramethyl rhodamine (TAMRA) and 4,7-dichlorotetramethylrhodamine (dTAMRA). Additional rhodamine dyes can be found, for example, in U.S. Pat. No. 5,366,860 (Bergot et al.), U.S. Pat. No. 5,847,162 (Lee et al.), U.S. Pat. No. 6,017,712 (Lee et al.), U.S. Pat. No. 6,025,505 (Lee et al.), U.S. Pat. No. 6,080,852 (Lee et al.), U.S. Pat. No. 5,936,087 (Benson et al.), U.S. Pat. No. 6,111,116 (Benson et al.), U.S. Pat. No. 6,051,719 (Benson et al.), U.S. Pat. Nos. 5,750,409, 5,366,860, 5,231,191, 5,840,999, and 5,847,162, U.S. Pat. No. 6,248,884 (Lam et al.), PCT Publications WO 97/36960 and WO 99/27020, Sauer et al., 1995, J. Fluorescence 5(3):247-261, Arden-Jacob, Neue Lanwellige Xanthen-Farbstoffe für Fluoresenzsonden und Farbstoff Laser, Verlag Shaker, Germany (1993), and Lee et al., Nucl. Acids Res. 20(10):2471-2483 (1992), Lee et al., Nucl. Acids Res. 25:2816-2822 (1997), and Rosenblum et al., Nucl. Acids Res. 25:4500-4504 (1997), for example. In one embodiment, the dye comprises a 4,7-dichloro-orthocarboxyrhodamine.

In some embodiments, the dye comprises a fluorescein dye, which comprises a fluorescein-type xanthene ring in which the C9 carbon atom is substituted with a phenyl or an orthocarboxy phenyl substituent (pendent phenyl group). An exemplary subset of fluorescein-type dyes are 4,7,-dichlorofluoresceins. Typical fluorescein dyes include, but are not limited to, 5-carboxyfluorescein (5-FAM), 6-carboxyfluorescein (6-FAM). Additional typical fluorescein dyes can be found, for example, in U.S. Pat. Nos. 5,750,409, 5,066,580, 4,439,356, 4,481,136, 4,933,471 (Lee), U.S. Pat. No. 5,066,580 (Lee), U.S. Pat. No. 5,188,934 (Menchen et al.), U.S. Pat. No. 5,654,442 (Menchen et al.), U.S. Pat. No. 6,008,379 (Benson et al.), and U.S. Pat. No. 5,840,999, PCT publication WO 99/16832, and EPO Publication 050684. In one embodiment, the dye comprises a 4,7-dichloro-orthocarboxyfluorescein.

In some embodiments, the dye comprises a cyanine, phthalocyanine, squaraine, or bodipy dye, such as described in the following references and references cited therein: U.S. Pat. No. 5,863,727 (Lee et al.), U.S. Pat. No. 5,800,996 (Lee et al.), U.S. Pat. No. 5,945,526 (Lee et al.), U.S. Pat. No. 6,080,868 (Lee et al.), U.S. Pat. No. 5,436,134 (Haugland et al.), U.S. Pat. No. 5,863,753 (Haugland et al.), U.S. Pat. No. 6,005,113 (Wu et al.), and WO 96/04405 (Glazer et al.).

In some embodiments, the dye comprises at least one donor and at least one acceptor (emitter) moiety in which the donor(s) provide excitation light to the acceptor(s), which then emit a fluorescent signal that is useful for detection. Examples of such dyes are described, for example, in U.S. Pat. Nos. 5,863,727, 5,800,996, 5,945,526, 6,335,440, and WO 01/16369.

In some embodiments, the invention comprises a method for detecting at least one target polynucleotide sequence in a sample. In some embodiments, the method comprises forming a ligation mixture comprising the sample and a plurality of different ligation probe sets, each ligation probe set comprising (a) a first ligation probe comprising a first target-specific portion, and (b) a second ligation probe comprising a second target-specific portion, wherein the first or second probe in each set further comprises an identifier tag portion that identifies the probe that contains the identifier tag portion, and wherein first and second target-specific portions of first and second ligation probes from at least one ligation probe set are hybridized to adjacent sequences in a target polynucleotide.

The ligation reaction mixture may be subjected to at least one cycle of ligation, thereby forming at least one first strand comprising the first target-specific portion, the second target-specific portion, and the identifier tag portion of at least one ligation probe set. In some embodiments, the plurality of different ligation probe sets comprises at least 10, 20, 30, 50 or 100 different ligation probe sets.

In some embodiments, a second strand is formed that is complementary to at least one first strand and that comprises sequence portions that are complementary to the first target-specific portion, the second target-specific portion, and the identifier tag of at least one ligation probe set.

Optionally, methods of the invention comprise amplifying the first strand, the second strand, or both.

Subsequently, one or more complexes are formed, wherein each complex comprises a first strand, a second strand, or both, and a mobility probe, said mobility probe comprising (a) a mobility defining moiety that imparts an identifying mobility or total mass to the mobility probe, and (b) a tag portion or tag portion complement, wherein the tag portion or tag portion complement is hybridized to the complementary tag portion complement or tag portion, respectively, in the first or second strand in one or more complexes. The one or more different mobility probes from the one or more complexes may then be released from the complex and may be detected using a mobility-dependent analysis technique (MDAT).

In some embodiments, at least one ligation probe set comprises at least two different first probes having target-specific portions that differ from each other by a single nucleotide base. In some embodiments, the different single nucleotide base is located at a terminus, or one base from the terminus, of the target-specific portion of the two different first probes, such that said terminus is adjacent the terminus of the target-specific portion of the second probe of the ligation probe set when the first and second ligation probes are bound to their complementary target sequences. In some embodiments, each different single nucleotide base is located within the target-specific portion of the different first probes. In some embodiments, at least one ligation probe set comprises at least two different first probes having target-specific portions that differ from each other by more than one nucleotide base.

In some embodiments, at least two different first probes of at least one ligation probe set comprise identical identifier tag portions. In some embodiments, at least two different first probes of at least one ligation probe set comprise different identifier tag portions.

In some embodiments, the target-specific portion of the first ligation probe in at least one ligation probe set is designed to be complementary to a target sequence that is downstream of the target sequence to which the second ligation probe in the at least one ligation probe set is complementary. In some embodiments, the target-specific portion of the first ligation probe in at least one ligation probe set is designed to be complementary to a target sequence that is upstream of the target sequence to which the second ligation probe in the at least one ligation probe set is complementary.

In some embodiments, the first and second target-specific portions of the first and second probes in each ligation probe set hybridize to immediately adjacent complementary target sequences, without a nucleotide gap. In some embodiments, the first and second probes in each ligation probe set are ligation competent. In other words, the first and second probes are suitable for ligation when hybridized to their adjacent complementary target sequences, without requiring further modification before chemical or enzyme-mediated ligation can occur. In other embodiments, first and second probes are ligation-incompetent, and require modification of at least one of the first or second probes to obtain a ligation-competent form. Thus, in some embodiments, the method comprises at least one cycle of ligation in which at least one ligation probe set is converted to a ligation-competent form to allow ligation.

In some embodiments, the first and second probes in each ligation probe set hybridize to adjacent complementary target sequences that are separated by a gap of no more than two nucleotides (i.e., by one or two nucleotides). Such probe sets are usually ligation-incompetent for enzyme-mediated ligation unless the gap is filled by a polymerase enzyme with a nucleotide or nucleotides that are complementary to the target sequence. After gap filling, the extended probe can be ligated with a ligase enzyme for example. Alternatively, such probe sets may be ligation-competent if they contain suitable functional groups that allow chemical ligation of first and second probes across the gap.

Thus, in some embodiments, the invention also encompasses ligation techniques such as gap-filling ligation, including, without limitation, gap-filling OLA and gap-filling LCR, bridging oligonucleotide ligation, and correction ligation. Descriptions of these techniques can be found, among other places, in U.S. Pat. No. 5,185,243, published European Patent Applications EP 320308 and EP 439182, and published PCT Patent Application WO 90/01069. As discussed above, chemical ligation may also be used, with or without a gap between the adjacent ends of the first and second probes.

A variety of other types of ligation-incompetent probe sets can be used in accordance with the invention. For example, in some embodiments, the target-specific portion of the first ligation probe in at least one ligation probe set is designed to be complementary to a target sequence that is downstream of the target sequence to which the second ligation probe in the at least one ligation probe set is complementary, such that the upstream and downstream target sequences overlap by at least one nucleotide, and at least one cycle of ligation-comprises cleaving a 5′ portion of the second ligation probe to produce a truncated second probe that is ligation-competent with respect to the first probe. In some embodiments, prior to the cleaving step, the second probe comprises a 5′ portion that is non-complementary to the target sequences to which the first and second target-specific portions are complementary. In some embodiments, the ligation product from these probe sets is amplified linearly, by repeated cycles of ligation. In some embodiments, the method further comprises formation of one or more second strands complementary to the first strand(s) (first ligation product), by including in the ligation mixture a second pair of ligation-incompetent probes which are complementary to the first and second ligation probes of each set, for exponential or asymmetric amplification of each target sequence and complement thereof. A detailed description of such ligation embodiments can be found in WO 02/02823, which is incorporated by reference in its entirety.

In some embodiments, the first probe comprises a 3′ portion that is non-complementary to the target sequences to which the first and second target-specific portions are complementary, and at least one cycle of ligation-comprises cleaving the 3′ non-complementary portion from the first ligation probe to produce a truncated first probe that is ligation-competent with respect to the second probe or truncated second probe, if present. Exemplary embodiments of this type of ligation can be found in U.S. Pat. No. 5,792,607. Additional ligation procedures involving conversion of ligase-incompetent probe pairs to ligation-competent pairs can be found in U.S. Pat. No. 5,516,663 (Backman), U.S. Pat. No. 5,427,930 (Birkenmeyer), and U.S. Pat. No. 5,573,907 (Carrino). The foregoing U.S. patents are incorporated herein by reference.

The present invention, in some embodiments, provides a method for detecting at least one target sequence in a sample. In the method, a sample that contains, or may contain, a plurality of target sequences is combined with a plurality of different probe sets. Each probe set comprises (a) a first probe comprising a first target-specific portion and a 5′ primer-specific portion, and (b) a second probe comprising a second target-specific portion and a 3′ primer-specific portion, wherein the first and second probes in each set are suitable for ligation together when hybridized to adjacent complementary target sequences. The first or second probe in each set further comprises an identifier tag portion that is between the primer-specific portion and the target-specific portion. The identifier tag portion identifies the probe that contains the identifier tag portion.

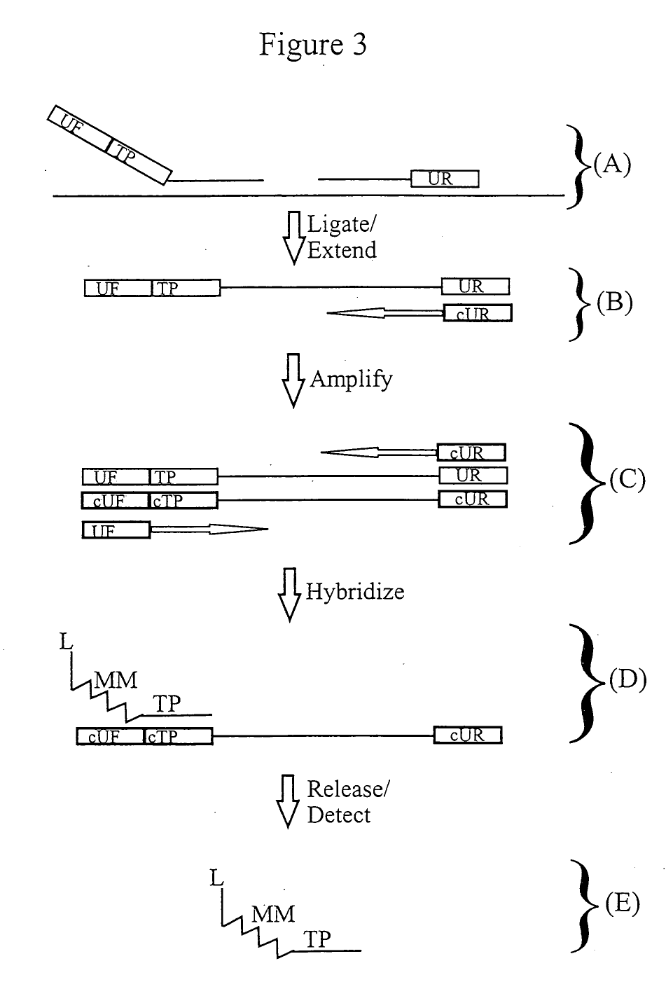

Various exemplary embodiments will now described with reference to FIGS. 3 through 5, which are provided solely for purposes of illustration and not to limit the invention.

In FIG. 3, a target sequence is treated with a plurality of different probe sets for the purposes of detecting a plurality of target nucleic acid sequences. FIG. 3(A) shows a probe set comprising a first probe and a second probe. The first probe comprises a first target-specific portion, an upstream tag portion, and a further upstream 5′ universal forward primer-specific portion. The second probe comprises a second target-specific portion and a downstream universal reverse primer-specific portion. For this example, the first probes in all probe sets contain the same universal 5′ “forward” primer-specific portion, and the second probes in all probe sets contain the same universal 3′ “reverse” primer-specific portion. Following hybridization, such probes can form a complex that is suitable for ligation. After ligation, the resulting ligation product comprises a 5′ primer-specific portion (UF), first and second target-specific portions, a 3′ primer-specific portion (UR), and an identifier tag portion (TP).

Following ligation, FIG. 3(B) shows a complementary universal reverse primer hybridized to the ligation product, for forming the complement of the ligation product by primer extension. Such extension allows subsequent exponential amplification of the ligation product when both universal primers are present, thereby forming a double stranded product comprising a first strand and a second strand that is hybridized to the first strand (FIG. 3(C)).

The amplification product can then be treated with a mobility probe comprising a mobility-defining moiety, a tag portion, and a label (FIG. 3(D)). The tag portion, which imparts an identifying mobility or total mass to the mobility probe, is hybridized to the tag portion complement in the amplified product. The hybridized (bound) mobility probe can be released and subsequently detected by virtue of the mobility probe's label and distinct mobility, thereby identifying the target sequence (FIG. 3(E)).

It will be recognized that variations of the scheme presented in FIG. 3 can be implemented. For example, unincorporated probes and primers can be removed at any of variety of stages, using various experimental techniques. In one embodiment, using an affinity capture technique, streptavidin-based capture of biotinylated probes can be performed prior to or following the ligation reaction. It will be appreciated that the upstream (first) probe, the downstream (second) probe, or both, can be biotinylated. In a further embodiment, streptavidin-based capture of biotinylated reverse probe can be performed prior to or after the ligation reaction. A further streptavidin-based capture can be performed following formation of the mobility probe complexes, thereby removing undesired reaction components.

In some embodiments, undesired or unreacted reaction components can be removed by size exclusion chromatography. For example, purification can be performed using Microcon-100 columns, which are commercially available from Millipore, Medford, Mass., by following the manufacturer's instructions. In some embodiments, undesired or unreacted reaction components can be removed by ion exchange procedures.

In some embodiments, an exonuclease that is specific for unhybrdized polynucleotides that have a free 3′ hydroxyl can be used to selectively degrade unwanted residual unligated probes and/or primers). In some embodiment, a blocking group may be attached to the 3′ end of a “downstream” ligation probe (whose 5′ end is to be ligated to the 3′ end of the “upstream” probe) to confer resistance to 3′OH-specific exonucleases, thereby allowing for the removal of unligated upstream probes (e.g., see Barany et al., WO97/45559).

In some embodiments, a 5′ single-strand specific exonuclease can be used to cleave residual second probes, whereas ligation products can be protected from 5′ exonuclease degradation due to the absence of a free 5′ phosphate group at the 5′ end of the first probes (and 5′ end of the ligation products). In some embodiments, primer extension is performed following the one or more ligation cycles to generate double-stranded ligation products and double stranded unligated downstream probes, thereby allowing a 5′ specific exonuclease possessing a preference of double-stranded nucleic acids to selectively destroy double-stranded unligated downstream probes.

In some embodiments, combinations of these exonuclease-mediated steps may be employed to achieve removal of unligated probes.

In some embodiments, removal of residual probes following ligation may be accomplished by diluting an aliquot of the ligation reaction into a PCR mixture.

In some embodiments, one or both ligation probes comprise deoxy-uracil instead of deoxy-thymidine, thereby rendering the probes sensitive to degradation by uracil N-glycosylase. By introducing a primer extension step following the ligation reaction, the uracil-bearing ligation products can be converted into deoxy-thymidine bearing products. Through the introduction of urcacil N-glycosylase the original probes can be destroyed (e.g., see Barany et al., WO97/45559).

It will be recognized that a variety of mobility-dependent analysis techniques (MDAT) may be employed for the purposes of measuring the mobility probe, such techniques including, but not limited to, electrophoresis, such as gel or capillary electrophoresis, HPLC, mass spectroscopy, including MALDI-TOF, gel filtration and chromatography.

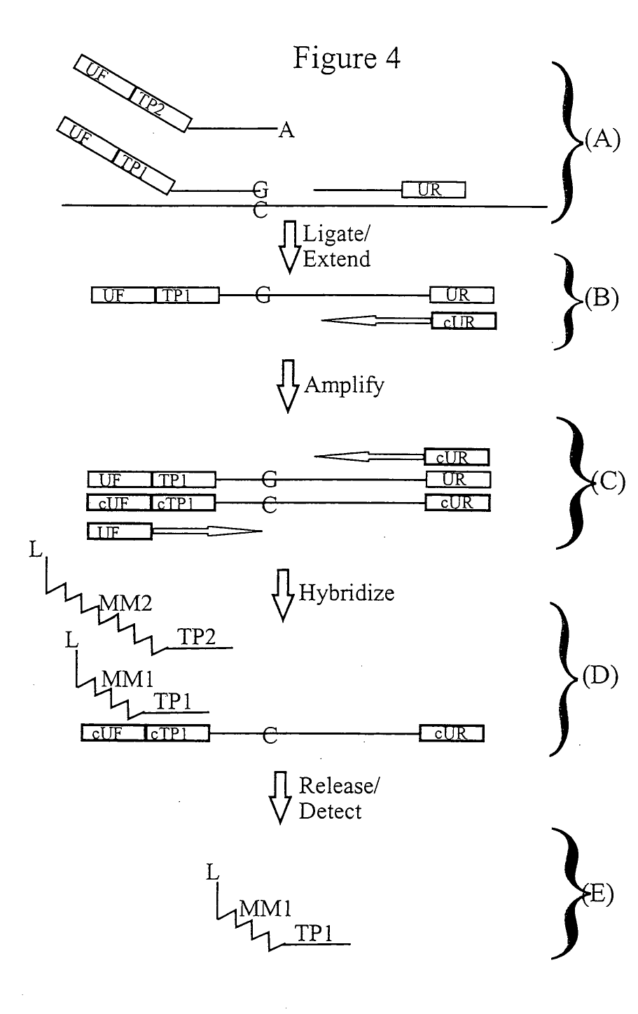

FIG. 4 illustrates how the procedure from FIG. 3 may be used to detect single nucleotide polymorphism (SNP) allelic variants. In FIG. 4(A), the probe set comprises a first probe and a second probe. The first probe comprises a first target-specific portion, an upstream tag portion, a further upstream 5′ universal forward primer-specific portion, and a polymorphic G nucleotide. In addition, the probe set also comprises a different first probe comprising a first target-specific portion, an upstream tag portion, a further upstream 5′ universal forward primer-specific portion, and a polymorphic A nucleotide. The second probe comprises a second target-specific portion and a downstream universal reverse primer-specific portion.

As shown, following hybridization, the complementary first probe that contains the G allele forms a ligation-competent complex with the target stand, but the A allele does not. Thereafter, the steps discussed in FIG. 3 are employed, resulting in detection of the target allele, which is homozygous in this example.

It will be recognized that the invention is not limited solely to genomic SNPs, but may also be practiced in the context of mRNA splice variant detection. In such embodiments, different mRNAs formed by alternative splicing (splice variants) can be reverse-transcribed into cDNA using methods well known in the art.

In addition to detection of sequence variants, some embodiments can be practiced to measure the relative expression levels of different mRNAs. In some embodiments, this may be accomplished by incorporating a promoter sequence, such as the promoter sequence for the T7 RNA polymerase. By incorporation into a ligation or amplification product, multiple rounds of T7 polymerase-mediated linear amplification can be performed, and the resulting amplification products can be detected and/or measured via hybridization, release, and detection of mobility probes.

In some embodiments, methods of the invention can be applied to multiple different samples for comparing levels of different target sequences that may be present in more than one of the different samples. Thus, the levels of a plurality of different target sequences can be compared and/or measured in the difference samples, to determine, for example, the relative expression levels of mRNA species or other nucleic acids of interest. For example, a first sample and a second sample may undergo steps of probe ligation, formation of second strand, optional amplification of first and/or second strand, complex formation, and release of different mobility probes, to produce a first mixture of different mobility probes from the first sample, and a second mixture of different mobility probes from the second sample. The mobility probes are chosen to provide uniquely identifiable mobilities or masses that correspond to each particular sample and the particular sequence target. In some embodiments, mobility probes may be used that contain the same tag or tag complement portion for the same target sequence to be detected, but the mobility probes comprise distinct mobilities (e.g., via distinct mobility defining entities) to identify the different sample sources. For example, to detect levels of a particular target sequence in two different samples, the mobility probes may comprise identical tag or tag complement portions but have slightly different mobilities or masses that are distinguishable by the selected mobility-dependent analysis technique. In electrophoretic detection, for example, the two mobility probes can elute near each other as pairs, providing a visual comparison of the ratio of the levels of the target sequence between the two samples. For example, for an embodiment in which sequences in two different samples are studied, mobility probes may be designed to produce two substantially identical patterns of peaks such that the elution times or rates of each pair of mobility probe peaks that correspond to the same target sequence from the two different samples are near but offset from each other by similar amounts. The mobility probes for each sample may also comprise the same label or different labels. Alternatively, for example, the mobility probes may have identical mobilities (and optionally may comprise identical tag or tag complement portions), but comprise distinguishable labels.

Further, in some embodiments, target sequences may be quantified. For example, one or more standard target sequences can be added to the samples prior to probe ligation, to provide a calibration curve or baseline measurement and range by which unknown amounts of target sequences can be determined.

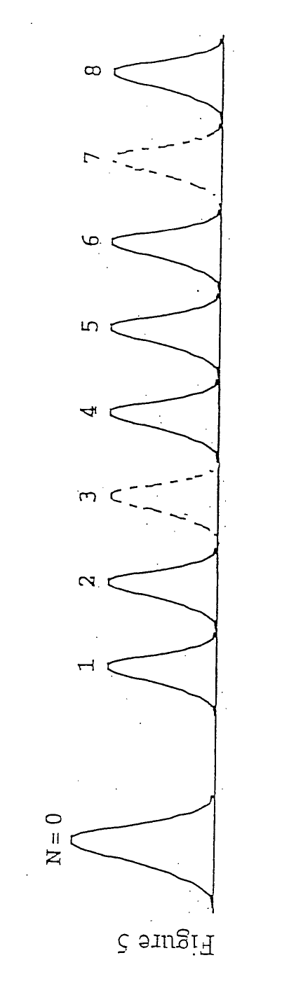

FIG. 5 shows an exemplary schematic electropherogram obtained by electrophoresis of a 8 different mobility probes. Observed peaks are shown by solid lines, and expected but absent peaks are shown by dashed lines. As can be seen, mobility probes number 1, 2, 4, 5, 6, and 8 are observed, indicating the presence of the corresponding target sequences in the sample. Peaks for mobility probes 3 and 7 are absent, indicating that the corresponding target sequences are absent from the sample or are present at levels too small to be detected. Detection may be accomplished manually or in automated fashion. Various instruments and protocols (e.g., GENEMAPPER™ software products from Applied Biosystems) are commercially available or have been published which are suitable for detection in accordance with the invention. In addition, a useful software approach for facilitating peak detection for electrophoretic detection is described in the co pending, commonly owned U.S. patent application filed concurrently herewith entitled “POLYNUCLEOTIDE SEQUENCE DETECTION ASSAYS AND ANALYSIS” U.S. application No. 60/445,494, which is incorporated herein by reference in its entirety. Additionally, previous U.S. provisional application 60/445,636 and 60/427,818 are also incorporated herein by reference.

The invention is further illustrated by way of the following example that is not intended to limit the invention in any way.

### EXAMPLE

A probe set is prepared for each target nucleic acid sequence, each set comprising first and second ligation probes designed to hybridize adjacently to the desired complementary target sequences. For a 5-plex assay, ten probes sets can be prepared for detecting five pairs of alternate alleles at five different loci. The ten probe sets include five pairs of locus-specific probe sets, wherein each pair is designed to detect two possible alternative single nucleotide polymorphisms (SNPs) in a particular locus.

The first probe in each set comprises a 5′ primer-specific portion at its 5′ end, a first target-specific portion at its 3′ end, and an identifier tag portion between the primer-specific portion and the target-specific portion. For this example, the sequence of the 5′ primer-specific portion of each of the one or more first probes is identical, and, for this example, has a length of 18-22 nucleotides and a Tm of 55-65° C.

The target specific portion of each first probe comprises a sequence that is complementary to a different target sequence in the sample. In this example, each pair of probe sets comprises two different first probes that contain identical target-specific portions except for the presence of a different 3′ terminal nucleotide, for hybridizing to either of two alternative SNPs at the same locus. The target-specific portions may be designed to have approximately the same Tm values, all within approximately 2 or 3° C. at 10 nM. Exemplary ranges are as follows: 42-44° C., 53-55° C., or 57-60° C. In some embodiments, target-specific portions are designed to be approximately 17-25 nucleotides in length.

The identifier tag portion in each first probe comprises a distinct sequence that can be used to identify the particular target-specific portion in the probe. In this example, the identifier tag portions are designed to have approximately the same Tm (65-68° C.) and are from 22-26 nucleotides in length.

The second probe in each set comprises a target-specific portion at its 5′ end and a 3′ primer-specific portion at its 3′ end. In this example, the first and second probes in each set are designed to hybridize to their complementary target sequences, such that the 3′ end of the first probe abuts the 5′ end of the second probe. The resulting “nick complex” can be ligated.

For this example, the sequence of the 3′ primer-specific portion of the second probes in each probe set is identical, and, for this example, has a length of 18-22 nucleotides and a Tm of 55-65° C. In some embodiments, the Tm of the 3′ primer-specific portion is about 3° C. higher than the Tm of the 5′ primer-specific portion.

The probe sets are combined with a genomic DNA sample from human blood (Coriell Institute for Medical Research, Camden, N.J.) to form a ligation reaction mixture (10 μL) comprising sample gDNA (10 ng/μL), 20 mM Tris-HCl, pH 7.6, 25 mM potassium acetate, 10 mM magnesium acetate, 10 mM DTT, 1 mM NAD, 0.1% Triton X-100, 10 nM of each first probe, 20 nM of each second probe, and 3 to 10 U ligase (from 0.12 to 1.0 U/μL) (Taq ligase mutant AK16D, Nucl. Acids Res. 27:788 (1999), or Taq ligase (New England BioLabs, Beverly, Mass.).

The ligation reaction mixture is pre-heated with a 9700 Thermocycler (Applied Biosystems, Foster City, Calif.) at 95° C. for 2 minutes, followed by 80° C. for 1 minute during which the ligase is added. Ligation products may be generated using thermocycling conditions of: 10-40 cycles at 90° C. for 10 seconds and 55-60° C. for 4 minutes. After the cycling, the mixture is optionally heated at 95° C. for 10-20 minutes. In another exemplary protocol, the reaction mixture is pre-heated at 90° C. for 3 minutes, followed by 10-40 thermocycles (90° C. for 15 seconds and 55° C. for 5 minutes), followed by heating at 95° C. for 10-20 minutes (optional) and a 4° C. hold.

Following ligation, streptavidin magnetic (SAV-Mag) beads can be used to select biotinylated ligation products. For example, the second probe in each probe set includes a 3′ biotin moiety for streptavidin capture. For example, 10 μL of SAV-Mag beads (10⁶-10⁷ beads/μL, 0.7 μm diameter, Seradyn, Indianapolis, Ind.) are added to the 10 μL ligation reaction mixture and incubated at 25° C. or ambient temperature for 10-30 minutes. After incubation, a magnet is placed at the bottom of the sample for 2 minutes, and the supernatant is removed by micropipette. The beads are then washed in 100 μL 1× phosphate buffered saline containing 0.1% Tween-20. After the wash, the magnet is then placed near the bottom of the sample for 2 minutes, and the supernatant is removed by micropipette.

Amplification can be performed by PCR by suspending the bead-immobilized ligation products in an amplification solution (10 μL) comprising 5 μL of Amplitaq Gold PCR Master Mix (Applied Biosystems)+5 μL water, and 1 μM each (final concentration) of first and second universal primers (the first primer is complementary to the complement of the 5′ primer-specific portions of each first probe, and the second primer is complementary to the 3′ primer-specific portions of each second probe).

Alternatively, if the sample was not purified using streptavidin bead capture, an amplification mixture can be prepared by transferring an aliquot of the ligation reaction mixture (1 μL) to an amplification solution (9 μL) to produce final concentrations as just described.

The amplification reaction mixture is pre-heated at 95° C. for 10 minutes, followed by 25-30 cycles using 92° C. for 15 seconds, 55° C. for 60 seconds, 72° C. for 30 seconds, ending with 72° C. for 7 minutes and 4° C. hold.

In some embodiments, a post-amplification purification is performed by adding 10 μL of SAV-Mag beads (10⁷ beads/μL, 0.7 μm diameter, Seradyn) to the 10 μL amplification reaction mixture and incubated at ambient temperature for 10-30 minutes. Next, 10 μL of 0.1 M NaOH is added and the resulting mixture is incubated at ambient temperature for 10-20 minutes. After the incubation, a magnet is placed near the bottom of the mixture for 0.5-2 minutes, and the supernatant is removed by micropipette.

For detection of the different amplified ligation products, mobility probes can be prepared for hybridization to the tag portions or tag portion complements of the amplified strands, such that each mobility probe can be used to identify a particular target sequence for which the corresponding probe set was successfully ligated and amplified. For example, each mobility probe can comprise a tag portion or tag portion complement comprising a polynucleotide sequence (e.g., 22-26 nt) that is specific for the corresponding tag portion or tag portion complement in one of the amplified strands. Each mobility probe additionally comprises a mobility defining moiety that imparts an identifying mobility (e.g., for electrophoretic detection) or total mass (for detection by mass spectrometry) to the mobility probe. For example, the mobility probe for each different target sequence may comprise a polyethylene glycol (PEO) polymer segment having a different length (EO)ₙ, where n ranges from 1 to 10. For fluorescence detection, the mobility probes may additionally include fluorescent dyes, such as FAM and VIC dyes, for detection of the different, alternative SNPs at each target locus. These may be attached by standard linking chemistries to the “5′ end” of the mobility defining moiety (the end of the mobility defining moiety that is opposite to end that is linked to the tag portion or tag portion complement).

The mobility probes may be hybridized to amplified strands as follow. To the bead-immobilized amplification products is added 10 μL of a mixture of mobility probes (final concentration 100 pM to 1 nM each, or 100 pM to 10 nM each, or 100 pM to 100 nM each) in 4×SSC buffer containing 0.1% SDS), and the resulting mixture is incubated at 50° C. 60° C. for 30 minutes. After the incubation, 100-200 μL 1×PBS buffer containing 0.1% Tween-20 is added. After the mixture is vortexed, a magnet is placed near the bottom of the mixture tube for 2 minutes, and the supernatant is removed by micropipette, and this process of adding PBS buffer, vortexing, and removing supernatant is repeated twice more. A final wash is performed with 0.1×PBS containing 0.1% Tween-20, followed by vortexing and removal of supernatant. To the beads are added 10 μL of DI-formamide solution (Applied Biosystems) and 0.25 μL of size standards (LIZ 120™, Applied Biosystems). The resulting mixture is heated to 95° C. for 5 minutes, and an aliquot is loaded by electrokinetic injection (30 sec at 1.5 kV) onto a 36 cm long capillary tube loaded with POP6™ (Applied Biosystems) on an ABI Prism 3100 Genetic Analyzer™, 15 kV run voltage, 60° C. for 20 minutes using a FAM™, VIC™ and LIZ™ Matrix (e.g., GENEMAPPER G5 Matrix, Applied Biosystems).

In the resulting electropherogram, fluorescent peaks are observed for different mobility probes, due to their distinct combinations of mobility and fluorescent label. The mobility and fluorescent signal for each mobility probe is usually already known from prior experimentation, so that the corresponding target sequences can be readily identified. In some embodiments, two different mobility probes may migrate with the same mobility, but they can be distinguished if they comprise different labels (e.g., FAM and VIC). In some embodiments, each mobility probe is designed to migrate with a distinct mobility, and the attached fluorescent label alternates between FAM and VIC for each successive peak, to further simplify identification of the probes. A size standard can also be used to facilitate identification of the probes.

All references cited herein are incorporated by reference for any purpose as if each was separately but expressly incorporated by reference.

Although the invention has been described with reference to particular embodiments, it will be appreciated that various changes and modifications may be made without departing from the scope and spirit of the invention.

1. A method for detecting at least one target polynucleotide sequence in a sample, the method comprising:
forming a ligation mixture comprising the sample and a plurality of different ligation probe sets, each ligation probe set comprising (a) at least one first ligation probe comprising a first target-specific portion, and (b) at least one second ligation probe comprising a second target-specific portion, wherein the first or second probes in each set further comprise an identifier tag portion that identifies the probe that contains the identifier tag portion, and wherein the first and second target-specific portions of the first and second ligation probes from at least one ligation probe set are hybridized to adjacent sequences on a target polynucleotide;
subjecting the ligation reaction mixture to at least one cycle of ligation, thereby forming at least one first strand comprising the first target-specific portion, the second target-specific portion, and the identifier tag portion of at least one ligation probe set;
forming one or more complexes, wherein each complex comprises a first strand hybridized to a mobility probe, said mobility probe comprising (a) a mobility defining moiety that imparts an identifying mobility to the mobility probe, and (b) a tag portion complement, wherein the tag portion complement is hybridized to the complementary tag portion on the first strand in one or more complexes;
releasing one or more different mobility probes from the one or more complexes, and
detecting the one or more released mobility probes using a mobility-dependent analysis technique (MDAT).

2. The method of claim 1 wherein at least one ligation probe set comprises at least two different first probes having target-specific portions that differ from each other by at least one nucleotide.

3. The method of claim 1, wherein the first and second probes in at least one ligation probe set hybridize to nearly adjacent complementary target sequences that are separated by a gap of at least one nucleotide, wherein the ligation mixture further comprises a polymerase and free nucleotides, whereby said is filled in prior to ligation.

4. The method of claim 1, wherein the second ligation probe comprises a 3′ primer-specific portion, and an amplification is performed comprising polymerase-mediated extension of a reverse primer, wherein the reverse primer is complementary to the 3′ primer-specific portion, to form a linearly amplified strand, wherein the tag portion of the mobility probe is hybridized to a tag portion complement in the linearly amplified strand.

5. The method of claim 1, wherein the first ligation probe comprises a 5′ primer-specific portion and the second ligation probe comprises a 3′ primer-specific portion, and an amplification reaction is performed comprising polymerase-mediated extension of a forward primer and a reverse primer, wherein the forward primer is complementary to the complement of said 5′ primer-specific portion and the reverse primer is complementary to the 3′ primer-specific portion, thereby forming exponentially amplified first strand and second strand.

6. The method of claim 5, wherein the plurality of different probe sets comprises at least one probe set comprising (i) first probes having a universal 5′ forward primer-specific portion, and (ii) second probes having a universal 3′ reverse primer-specific portion, and said amplifying comprises amplifying first and second strands of the at least one probe sets using a single universal primer set comprising a universal forward primer and a universal reverse primer.

7. The method according to claim 1, wherein the at least one first probe or the at least one second probe, or both, further comprise an affinity moiety, said method further comprising;
contacting the first strand with a binding moiety,
immobilizing the first strands,
removing un-immobilized reaction components,
hybridizing at least one mobility probe to the remaining immobilized strands,
removing unhybridized mobility probes,
eluting the bound mobility probes, and,
determining the identity of the at least one target polynucleotide sequence by the identity of the mobility probe in a mobility dependent analysis technique.

8. The method according to claim 7 wherein the affinity moiety is biotin.

9. The method according to claim 1 further comprising, incorporating an affinity moiety into the at least one target polynucleotide sequence, immobilizing the at least one target polynucleotide sequence with a binding moiety specific for the affinity moiety,
hybridizing the at least one immobilized target polynucleotide sequence with the one or more probe sets, and,
removing unhybridized probes prior to the ligation reaction.

10. The method according to claim 1 wherein the 5′ portion of the upstream ligation probe and/or the 3′ portion of the downstream ligation probe are resistant to nuclease digestion, wherein unligated probes are removed with 3′ acting and/or 5′-acting exonucleases.

11. The method according to claim 1 wherein the mobility probes further comprise a label.

12. The method according to claim 11 wherein the label further comprises a fluorophore.

13. The method according to claim 1 wherein the mobility modifier of the mobility probe is chosen from at least one of the following; polyethylene oxide, polyglycolic acid, polylactic acid, polypeptide, oligosaccharide, and polyurethane, polyamide, polysulfonamide, polysulfoxide, and block copolymers thereof, including polymers composed of units of multiple subunits linked by charged or uncharged linking groups.

14. The method according to claim 1 wherein the mobility dependent analysis technique further comprises capillary electrophoresis.

15. The method according to claim 1 wherein the mobility dependent analysis technique further comprises mass spectrometry.

16. A method for detecting at least one target polynucleotide sequence in a sample, the method comprising:
forming a ligation mixture comprising the sample and a plurality of different ligation probe sets, each ligation probe set comprising (a) at least one first ligation probe comprising a first target-specific portion, and (b) at least one second ligation probe comprising a second target-specific portion, wherein the first or second probe in each set further comprises an identifier tag portion that identifies the probe that contains the identifier tag portion, and wherein first and second target-specific portions of first and second ligation probes from at least one ligation probe set are hybridized to adjacent sequences in a target polynucleotide;
subjecting the ligation reaction mixture to at least one cycle of ligation, thereby forming at least one first strand comprising the first target-specific portion, the second target-specific portion, and the identifier tag portion of at least one ligation probe set;
forming a second strand that is complementary to at least one first strand and that comprises sequence portions that are complementary to the first target-specific portion, the second target-specific portion, and the identifier tag of at least one ligation probe set;
amplifying the first strand and the second strand by a PCR,
forming one or more complexes, wherein each complex comprises a first strand or a second strand and a mobility probe, said mobility probe comprising (a) a mobility defining moiety that imparts an identifying mobility or total mass to the mobility probe, and (b) a tag portion or tag portion complement, wherein the tag portion or tag portion complement is hybridized to the complementary tag portion complement or tag portion, respectively, in the first or second strand in one or more complexes;
releasing one or more different mobility probes from the one or more complexes, and,
detecting the one or more released mobility probes using a mobility-dependent analysis technique (MDAT).

17. The method of claim 16 wherein at least one ligation probe set comprises at least two different first probes having target-specific portions that differ from each other by a single nucleotide base.

18. The method of claim 16, wherein the first and second probes in at least one ligation probe set hybridize to nearly adjacent complementary target sequences that are separated by a gap of at least one nucleotide, wherein the ligation mixture further comprises a polymerase and free nucleotides, whereby said gap can be filled in prior to ligation.

19. The method of claim 16, wherein the first ligation probe comprises a 5′ primer-specific portion and the second ligation probe comprises a 3′ primer-specific portion, and the amplification reaction is performed comprising polymerase-mediated extension of a forward primer and a reverse primer, wherein the forward primer is complementary to the complement of said 5′ primer-specific portion and the reverse primer is complementary to the 3′ primer-specific portion, to form exponentially amplified first strand and second strand.

20. The method of claim 16, wherein the plurality of different probe sets comprises at least one probe set comprising (i) first probes having the same 5′ forward primer-specific portion, and (ii) second probes having the same 3′ reverse primer-specific portion, and said amplifying comprises amplifying first and second strands of the at least one probe sets using a single universal primer set comprising a universal forward primer and a universal reverse primer.

21. The method according to claim 20, wherein the 5′ end of the at least one universal reverse primer comprises an affinity moiety, said method further comprising
contacting the amplified first and second strands with a binding moiety,
immobilizing the amplified strands,
removing un-immobilized reaction components,
eluting and removing the strand lacking the affinity moiety,
hybridizing at least one mobility probe to the remaining immobilized strands,
removing unhybridized mobility probes,
eluting the bound mobility probes, and,
determining the identity of the at least one target polynucleotide sequence by the identity of the mobility probe in a mobility dependent analysis technique.

22. The method according to claim 21 wherein the affinity moiety is biotin.

23. The method according to claim 16 further comprising,
incorporating an affinity moiety into the at least one target polynucleotide sequence,
immobilizing the at least one target polynucleotide sequence with a binding moiety specific for the affinity moiety,
hybridizing the at least one immobilized target polynucleotide sequence with the one or more probe sets, and,
removing unhybridized probes prior to the ligation reaction.

24. The method according to claim 16 wherein the 5′ portion of the upstream ligation probe and/or the 3′ portion of the downstream ligation probe are resistant to nuclease digestion, wherein unligated probes are removed with 3′ acting and/or 5′-acting exonucleases.

25. The method according to claim 16 wherein the mobility probes further comprises a label.

26. The method according to claim 25 wherein the label further comprises a fluorophore.

27. The method according to claim 16 wherein the mobility modifier of the mobility probe is chosen from at least one of the following, polyethylene oxide, polyglycolic acid, polylactic acid, polypeptide, oligosaccharide, and polyurethane, polyamide, polysulfonamide, polysulfoxide, and block copolymers thereof, including polymers composed of units of multiple subunits linked by charged or uncharged linking groups.

28. The method according to claim 16 wherein the mobility dependent analysis technique further comprises capillary electrophoresis.

29. The method according to claim 16 wherein the mobility dependent analysis technique further comprises capillary electrophoresis.
